(19) United States
(12) Patent Application Publication
(10) Pub. No.: US 2026/0283838 A1
(43) Pub. Date: Sep. 24, 2026

(54) VAGINAL TOOL AND METHOD

(71) Applicant: Mary Elizabeth Schofield, Mineral Wells, TX (US)

(72) Inventor: Mary Elizabeth Schofield, Mineral Wells, TX (US)

(21) Appl. No.: 19/573,822

(22) Filed: Mar. 20, 2026

Related U.S. Application Data
(60) Provisional application No. 63/775,904, filed on Mar. 21, 2025.

(51) Int. Cl. A61F 5/455

(52) U.S. Cl. CPC A61F 5/4553

(57) ABSTRACT

A vaginal tool for the removal of a menstrual cup from the vaginal canal includes a handle portion, a neck portion, and a hook portion. The handle portion has a cylindrical shape and is formed to include a longitudinal arc. The neck portion is coupled to the handle portion and includes a planar surface. The hook portion is coupled to the neck portion opposite the handle portion. The hook portion has a hook extending outward beyond the handle portion over the planar surface. A method of use of the vaginal tool includes inserting the vaginal tool into the vaginal canal with the hook entering first. Passing the hook beyond the menstrual cup so as to break the seal of the menstrual cup with the vaginal wall. Then pulling downward to engage the hook with the menstrual cup and slide the menstrual cup out though the vaginal canal.

### CROSS REFERENCE TO RELATED APPLICATIONS

This application claims the benefit of an earlier filing date and right of priority to U.S. Provisional Application No. 63/775,904, filed 21 Mar. 2025, the contents of which is incorporated by reference herein in its entirety.

### BACKGROUND OF THE INVENTION

### 1. Field of the Invention

The present application relates to a medical device, and more particularly to a tool to assist in vaginal health.

### 2. Description of Related Art

Menstrual cups are small flexible devices that are inserted into the vaginal canal of a woman. The cup is configured to catch or collect the woman's period flow during a menstrual cycle. These act differently from tampons or pads which are configured to absorb the period flow. The period flow is collected within the cup overtime. The cup may be pulled out of the vaginal canal to drain the period flow and is suitable for reuse after washing.

In order to operate correctly, the menstrual cup is configured to seal inside the vaginal canal. A suction is formed against the walls of the vaginal canal that prevents leakage of the period flow around the menstrual cup. While this may seem ideal, disadvantages of having this suction exist. Predominantly, this suction makes it difficult to remove the menstrual cup. Although a tab is located at a bottom portion of the menstrual cup, grabbing hold of it and pulling the menstrual cup is problematic and can be difficult to break the suction.

A few different methods of tools have been provided to try and assist in removing menstrual cups. One such device is in essence in a long gated stick with a spoon like end that is configured to be inserted into the vaginal canal and slid alongside a lower portion of the menstrual cup. The tool takes advantage of the flexible nature of the menstrual cup and is designed to push inward on the menstrual cup so as to break the seal it has with the vaginal wall. Naturally, this process could lead to spillage of period flow and can actually be extremely difficult to maintain the release of suction at the same time as pulling out the menstrual cup. Users often find this to be awkward and struggle to accomplish it.

Another type of tool uses finger sleeves that sit on your fingers and has a claw like tip. The user puts these on their fingers and inserts the fingers into the vaginal canal with the hope of moving them like scissors so as to engage the tips onto the bottom of the menstrual cup. If the user is successful, a better grasp of the menstrual cup may be obtained however this is difficult to accomplish when the user cannot see what they are doing.

With both of these tools, a user is left with many disadvantages and difficulties in trying to remove the menstrual cup. Although strides have been made, shortcomings remain. It is desired that an assembly be provided that allows a user to simultaneously break the seal of the menstrual cup and provide a downward force while preventing the menstrual cup to reseal.

### BRIEF SUMMARY OF THE INVENTION

It is an object of the present application to provide a vaginal tool configured to remove menstrual cups from the vaginal canal of a woman. An object of the tool is to easily slide within the vaginal canal between the vaginal wall and the menstrual cup, and then permit its rotation to hook over the top of the menstrual cup to permit a downward pull and subsequent removal. A further object of the tool is to pass beyond the rim of the menstrual cups and to break the seal of the cup.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as

limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a vaginal tool according to an embodiment of the present application.

FIG. 2 is a side view of the tool of FIG. 1.

FIG. 3 is a front view of the tool of FIG. 1.

FIGS. 4-5 are front views of an exemplary of a woman's reproductive organs using the tool of FIG. 1 to remove a menstrual cup.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

The vaginal tool and method of use of the present application is configured to simplify and make easier the removal of menstrual cups from the vaginal canal of a woman. The tool is configured to slide within the vaginal canal between the vaginal wall and the menstrual cup the tool passes beyond the rim of the menstrual cups so as to break the seal the tool is rotated and is brought into contact with the top of the menstrual cup rim allowing the person to pull down while maintaining a broken seal thereby making removal easier and less messy.

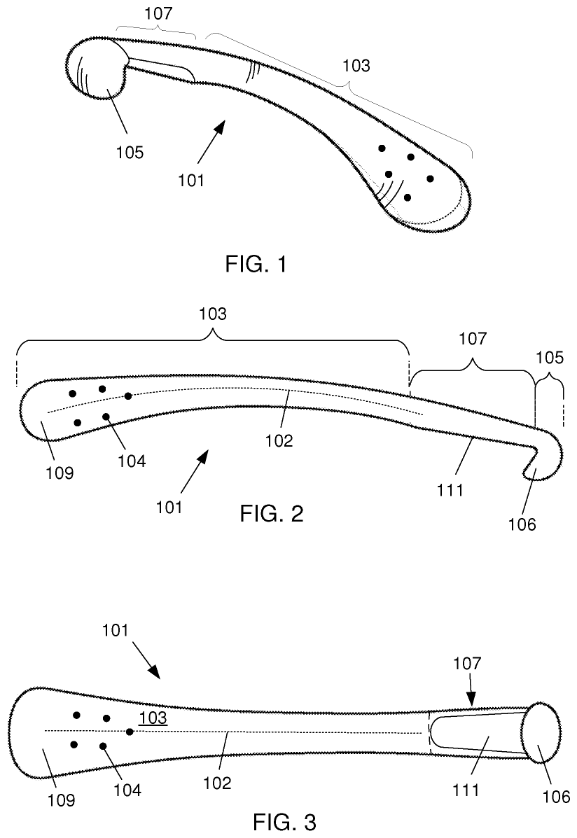

Referring now to FIGS. 1-3 in the drawings, views of vaginal tool 101 are provided. Tool 101 is an elongated stick like object including a handle portion 103 and a hook portion 105. The neck portion 107 bridges and connects handle portion 103 with hook portion 105. Handle portion 103 is configured to gradually taper inward so as to seamlessly join and form with neck portion 107. Handle portion 103 has a gradual longitudinal arc 102 seen most clearly in FIG. 2. FIG. 2 illustrates a side view (along a plane) defined by the curve of the longitudinal arc 102 of handle portion 103. The hook portion 105 extends away from the neck portion 107 along the same plane that is defined by the longitudinal arc 102, seen in FIG. 2. The hook portion 105 includes hook 106.

The form of tool 101 allows a user to grasp near the bulbous distal end 109 of portion 103 and angle tool 101 through the vaginal canal with ease. It is understood that handle portion 103 may include a surface treatment 104 that is configured to enhance grip. Surface treatment 104 may be in the form of dimples or bumps on the surface, for example, although other grip enhancing surface treatments are considered suitable. Surface treatment 104 may be used at any location along the surface of body portion 103.

Neck portion 107 is configured to include a narrowed shape and form compared to handle portion 103, wherein the diameter of the neck portion 107 is on average smaller than that of the handle portion 103. Neck portion 107 is configured to include a planar surface 111 that is adjacent to and beneath the hook 106. It is understood that tool 101 as a whole is configured to maintain a streamlined and compact shape so as to minimize interference or bulkiness when traveling through the vaginal canal. The use of planar surface 111 helps to minimize the size of neck portion 107, and to allow it to contact the curved menstrual cup with more stability along planar surface 111. Hook 106 extends outward over planar surface 111.

Hook portion 105 is formed from neck portion 107. Hook portion 105 includes hook 106 which extends outward beyond handle portion 103 and neck portion 107. Hook portion 105 is generally elongated wherein its width is longer from the side as it is from the front. This is seen predominantly in FIG. 2, wherein the side view of hook portion 105 is illustrated. The elongated nature of portion 105 allows tool 101 to form and define the hook 106 of hook portion 105. In FIG. 3, wherein a front view is illustrated, the general width of hook portion 105 is similar to that of neck portion 107.

During insertion, tool 101 is inserted within the vaginal canal wherein the sides of hook portion 105 slide between the menstrual cup and the vaginal wall. This can be referred to as hook portion 105 being parallel with the vaginal wall, such that hook 106 is generally orthogonal to that of the menstrual cup 99. This is in contrast to having the front and back portions of tool 101 slide in contact with a menstrual cup and vaginal wall, wherein hook portion 105 would be considered to be perpendicular with the vaginal wall. The elongated nature at its side allows it to more easily translate between menstrual cup 99 and the vaginal wall with minimal disruption.

Tool 101 may be formed and made from various different materials. Ideally medical grade silicone is preferred however it is understood that other processes or materials may be selected. Tool 101 is configured to alleviate the issues and disadvantages of legacy tools on the market. The length of tool 101 may be such as to allow the user to keep fingers external to the vaginal opening so as to minimize the chance of infection and increase cleanliness. The precise size of handled portion 103 can be selected based upon various design constraints.

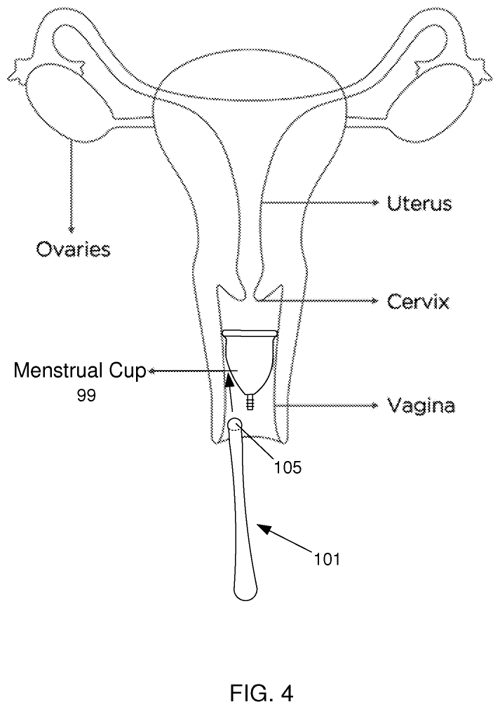

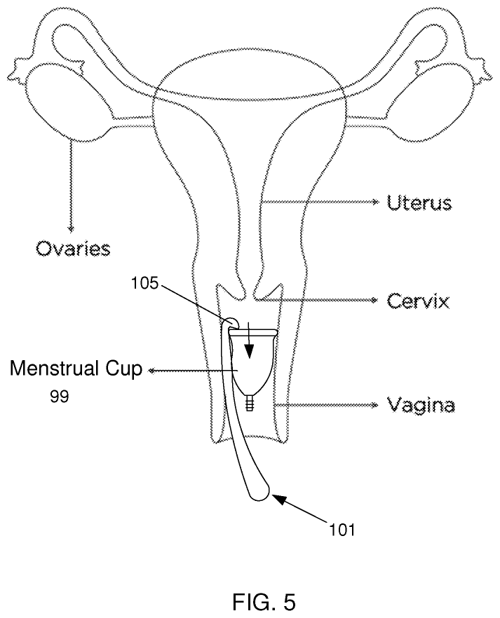

Referring now also to FIGS. 4 and 5 in the drawings, front exemplary views of a woman's reproductive anatomy are provided and illustrated with tool 101. FIGS. 4 and 5 are used to illustrate the method of operation or use of tool 101 with respect to the woman. In the vaginal opening is located a representative menstrual cup. In FIG. 4, the menstrual cup 99 is properly seated and sealed within the vagina. As noted previously two primary disadvantages of legacy tools are 1) the inability to maintain seal breakage of menstrual cup 99 during removal; and 2) an inability to cleanly grasp or hold on to the menstrual cup in the vaginal canal during removal.

Tool 101 is inserted through the vagina along the vaginal wall adjacent menstrual cup 99. Hook 106 is inserted first. During insertion, hook portion 105 slowly separates cup 99 from the vaginal wall. Tool 101 is oriented to be parallel to the vaginal wall to avoid interference with hook 106 and the sides of the menstrual cup 99. Tool 101 is inserted passed or beyond an upper rim of cup 99 so as to break the seal created with the vaginal wall and to locate hook 106 above cup 99. Tool 101 is rotated in the vagina such that hook 106 extends over a portion of cup 99 (see FIG. 5). The user then applies a downward force upon tool 101 and slides menstrual cup 99 out of the vaginal canal.

A user may optionally also shift tool 101 around the cup 99 before the step of pulling or during pulling on cup 99. The act of shifting may help to maintain the upright orientation of cup 99 during removal. It is understood that the need for rotation is not always necessary and that tool 101 may be inserted being perpendicular to the vaginal wall in some instances (hook 106 facing cup 99). With either insertion method, tool 101 is able to accomplish its designed function.

A big advantage of tool 101 compared to legacy devices is that tool 101 is configured to maintain the broken seal during removal as its insertion between the vaginal wall and the rim of cup 99 prevents the resealing of cup 99 in the vaginal canal. Secondly, tool 101 provides a secure and easy way to apply downward force on cup 99. The use of hook portion 105 avoids the need to pinch or squeeze areas of menstrual cup 99 which can be difficult to grab and hold on to. The current application has many advantages over the prior art.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A vaginal tool for the removal of a menstrual cup, comprising:
a body having:
a handle portion;
a neck portion coupled to the handle portion; and
a hook portion coupled to the neck portion opposite the handle portion, the hook portion defining a hook extending outward beyond the handle portion.

2. The tool of claim 1, wherein the handle portion formed to have a longitudinal arc.

3. The tool of claim 1, wherein the handle portion has a cylindrical form with a bulbous distal end.

4. The tool of claim 1, wherein the handle portion includes a surface treatment configured to enhance grip.

5. The tool of claim 1, wherein the neck portion includes a planar surface adjacent to the hook portion and beneath the hook.

6. The tool of claim 5, wherein the planar surface extends between the hook and the handle portion.

7. A vaginal tool for the removal of a menstrual cup, comprising:
a handle portion having a cylindrical shape and formed to include a longitudinal arc;
a neck portion coupled to the handle portion, the neck portion including a planar surface; and
a hook portion coupled to the neck portion opposite the handle portion, the hook portion defining a hook extending outward beyond the handle portion over the planar surface.

8. The tool of claim 1, wherein the planar surface extends between the hook and the handle portion.

9. The tool of claim 1, wherein the handle portion has a bulbous distal end.

10. The tool of claim 10, wherein the handle portion includes a surface treatment configured to enhance grip.

11. A method of removing a menstrual cup, comprising:
obtaining a vaginal tool of claim 2;
inserting the vaginal tool into the vaginal canal with the hook entering first;
passing the menstrual cup so as to break the seal of the menstrual cup with the vaginal wall, the hook extending above the menstrual cup; and
pulling downward to engage the hook with the menstrual cup and slide the menstrual cup out though the vaginal canal.

12. The method of claim 11, further comprising:
orienting the vaginal tool to be parallel to the vaginal wall; and
rotating the vaginal tool to be perpendicular to the vaginal wall with the hook extending over a portion of the menstrual cup.

13. The method of claim 12, wherein the step of rotating the vaginal tool to be perpendicular with the menstrual cup locates the planar surface so as to face the menstrual cup.

14. The method of claim 11, further comprising:
shifting the vaginal tool around the vaginal canal after breaking the seal to aid in removal of the menstrual cup.

15. The method of claim 14, wherein the vaginal tool is kept perpendicular to the menstrual cup when shifting.
